(19) United States
(12) Patent Application Publication
(10) Pub. No.: US 2005/0276848 A1
(43) Pub. Date: Dec. 15, 2005

(54) Sustained release neutralized divalproex sodium

(76) Inventors: Nilobon Podhipleux, Weston, FL (US); Xiu Xiu Cheng, Weston, FL (US); Unchalee Lodin, North Miami Beach, FL (US); Chih-Ming Chen, Taipei (TW); Avinash Nangia, Weston, FL (US); Dacheng Tian, Miramar, FL (US)

Correspondence Address: DAVIDSON, DAVIDSON & KAPPEL, LLC, 14th Floor, 485 Seventh Avenue, New York, NY, 10018, US

(21) Appl. No.: 10/867,799

(22) Filed: Jun. 15, 2004

(51) Int. Cl. A61K 9/22; A61K 9/20; A61K 31/19

(52) U.S. Cl. 424/468; 514/557

(57) ABSTRACT

The present invention is directed to sustained release oral dosage forms comprising neutralized divalproex sodium, methods of manufacturing the dosage forms, and methods of treatment with the dosage forms.

### FIELD OF THE INVENTION

The present invention is related to a sustained release oral dosage form suitable for once-a-day administration and comprising neutralized divalproex sodium. The present invention is further directed to a process for preparing a sustained release oral dosage form comprising neutralized divalproex sodium.

### BACKGROUND OF THE INVENTION

Valproic acid, or 2-propylpentanoic acid, and its salts and derivatives are compounds with anticonvulsant properties. Of these, valproic acid and its sodium salt (sodium valproate) are the most well known. U.S. Pat. No. 3,325,361 describes the use of valproic acid, sodium valproate and other salts and derivatives of valproic acid as anti-convulsants.

It has been recognized by those skilled in the art that both valproic acid and sodium valproate are difficult to formulate into solid oral dosage forms. Valproic acid, for example, is an oily liquid. Sodium valproate is known to be very hygroscopic and to liquify rapidly, and is, therefore, difficult to formulate into tablets.

Efforts have been made to address the problems associated with formulating valproic acid and sodium valproate into solid oral dosage forms. U.S. Pat. No. 5,049,586 (Ortega, et al.) describes valproic acid tablets having a specific composition, which tablets are said to be stable. The tablets contain valproic acid, magnesium oxide, corn starch, poyvinylpyrrolidone, sodium carboxymethylcellulose, and magnesium stearate in specific proportions.

U.S. Pat. No. 5,017,613 (Aubert, et al.) describes a process for preparing a composition containing valproic acid in combination with valproate sodium, wherein the process does not use any binder or granulating solvent. In the process, a mixture of valproic acid and ethylcellulose is prepared and valproate sodium is added to the mixture to form drug granules in the absence of any binder or granulating solvent. Precipitated silica is added to the granules before the compression into tablets.

Efforts have also been made to overcome the limited utility of valproic acid and sodium valproate in formulating solid dosage forms by creating a different salt form or a derivative of valproic acid. U.S. Pat. No. 4,895,873 (Schafer) describes a crystalline calcium salt of valproic acid, in which five valproic acid radicals are associated with one calcium ion. The crystalline salt, called calcium pentavalproate, is said to be non-hygroscopic.

U.S. Pat. No. 4,558,070 (Bauer, et al.) describes potassium, cesium or rubidium salt of valproic acid, which is prepared by combining 4 moles of valproic acid with 1 mole of the potassium, cesium or rubidium. U.S. Pat. No. 4,699,927 (Deboeck) describes arginine, lysine, histidine, ornithine or glycine salts of valproic acid.

U.S. Pat. Nos. 5,212,326 and 4,988,731 (Meade) describe divalproex sodium and its preparation. Divalproex sodium is described as an ionic oligomer in which one mole each of the valproic acid form coordinate bonds with the sodium of the sodium valproate molecule, where the valproate ion is ionically bonded to the sodium ion. Meade also describes the oligomeric compound as having better physical properties than either monomer from which it is made in that the oligomer is a crystalline, non-hygroscopic, stable solid compound.

Some patents describe sustained release dosage forms for divalproex sodium, valproic acid, its salts, amides, or other derivatives. U.S. Pat. No. 5,980,943 (Ayer, et al.) describes a sustained release delivery device for administering divalproex sodium, valproic acid, and its salts and derivatives. The device comprises a semipermeable wall containing drug granules that are microencapsulated with polyalkylene oxide or carboxymethylcellulose polymer.

U.S. Pat. No. 4,913,906 (Friedman, et al.) describes a controlled release dosage form containing divalproex sodium, valproic acid, valpromide and other valproic acid salts and derivatives. The composition is prepared by mixing the drug with hydroxypropyl cellulose, ethylcellulose, or esters of acrylic and methacrylic acid, and by applying high pressure to the mixture of the ingredients.

U.S. Pat. No. 5,807,574 (Cheskin, et al.) describes a controlled release dosage form containing divalproex sodium and a process for its preparation. The process involves melting divalproex sodium and mixing it with a molten wax to form a divalproex sodium-wax composite. The drug-wax mixture is formulated into a capsule.

U.S. Pat. No. 5,169,642 (Brinker, et al.) describes a sustained release dosage form containing granules of divalproex sodium, valproic acid or amides or esters or salts thereof and a polymeric viscosity agent. The drug is coated with a sustained release composition comprising specified portions of ethylcellulose or a methacrylic methylester, plasticizer, and detacifying agent.

U.S. Pat. No. 5,068,110 (Fawzi, et al.) describes various delayed-release tablets and capsules currently marketed, including the delayed-release divalproex sodium tablets manufactured by Abbott Laboratories, and states that the stability of an enteric coated capsules is increased by the application of thicker, higher levels of the enteric coating having a thickness of 14 mg/cm² to 24 mg/cm², alone or in combination with a hydroxypropylcellulose, hydroxymethylcellulose or hydroxypropylmethyl cellulose coating.

Divalproex sodium is a oligomer having a 1:1 molar ratio of sodium valproate and valproic acid. The oligomer is described as a stable crystalline solid and is designated as sodium hydrogen bis (2-propyl pentanoate).

Upon administration, divalproex dissociates into valproate ion in the gastrointestinal tract, and in that form exerts its pharmacological effect. Divalproex sodium is indicated for the treatment of patients with complex partial seizures, as well as for the treatment of mania associated with bipolar disorders and for prophylaxis of migraine headaches.

U.S. Pat. No. 4,558,070 (Bauer, et al.) indicates that divalproex sodium is a highly stable, non-hygroscopic, crystalline compound. Bauer also discusses a theory behind the stability of divalproex sodium, stating that it is not a mixture of the two precursors but a chemical entity, and that in the oligomer, the outer shell of electrons of the sodium atom is filled by coordination to the oxygen atoms of both valproic acid and valproate ions, resulting in a stable complex where the sodium ion is completely surrounded by oxygen. Bauer, et al., therefore, appears to indicate that the particular oligomeric structure and the molar ratio of divalproex sodium accounts for the stability of the compound.

Accordingly there exists a need for sustained release oral dosage forms comprising neutralized divalproex sodium such that e.g., divalproex sodium is capable of being formulated in the dosage form in the absence of the oligomeric structure and the equimolar ratio of sodium valproate and valproic acid.

### OBJECTS AND SUMMARY OF THE INVENTION

It is an object of the present invention to provide a neutralized divalproex sodium sustained release oral solid dosage form suitable for once-a-day administration.

It is an object of certain embodiments of the present invention to provide a neutralized divalproex sodium sustained release oral solid dosage form which provides a therapeutic effect up to about 24 hours after administration.

It is an object of certain embodiments of the present invention to provide a process for preparing a neutralized divalproex sodium sustained release oral solid dosage suitable for once-a-day administration.

It is an object of certain embodiments of the present invention to provide a once-a-day neutralized divalproex sodium method of treatment by orally administering a neutralized divalproex sodium sustained release oral solid dosage form on a once-a-day basis to a patient in need of such treatment.

In accordance with the above-mentioned objects of the invention, the present invention is directed in part to an oral solid dosage form comprising neutralized divalproex sodium and a sustained release material, said oral solid dosage form suitable for once-a-day administration. In certain preferred embodiments, the oral solid dosage form provides a therapeutic effect up to about 24 hours after administration.

In certain embodiments, the present invention is further directed to an oral solid dosage form comprising: granules of neutralized divalproex sodium and a pharmaceutically acceptable carrier said granules; a coating comprising a hydrophobic material coated over said granules; and a sustained release excipient; said oral solid dosage being suitable for once-a-day administration.

In certain embodiments, the present invention is further directed to an oral solid dosage form comprising a compressed tablet core comprising a neutralized divalproex sodium, a pharmaceutically acceptable carrier, a hydrophobic material, and a sustained release excipient; said compressed tablet core being overcoated with an enteric coating.

In preferred embodiments, the divalproex sodium used in the present invention is not present as an oligomeric structure or a 1:1 molar ratio of sodium valproate to valproic acid.

In preferred embodiments, the sustained release neutralized divalproex sodium oral dosage form of the present invention provides a sustained release of valproate ion when the dosage form is orally administered to human patients, preferably providing a therapeutic effect for about 24 hours after administration.

In certain embodiments, the present invention is further directed to a process for preparing a sustained release oral solid dosage forms comprising divalproex sodium; said process comprising preparing a neutralized divalproex sodium solution by combining divalproex sodium, having a valproic acid moiety and a sodium valproate moiety, with a base (e.g., sodium hydroxide) and an aqueous solvent. The base is added in sufficient amount to ensure neutralization of the valproic acid moiety of the divalproex sodium. In the neutralized divalproex sodium solution, divalproex sodium is not present as its oligomeric structure or the 1:1 molar ratio of sodium valproate and valproic acid. The valproic acid of the divalproex sodium is neutralized. Preferably the neutralized divalproex sodium solution contains from about 20 to about 60% valproic acid activity. The neutralized divalproex sodium solution is then incorporated in a sustained release oral solid dosage form with a sustained release material.

In certain embodiments, the process for preparing a sustained release oral solid dosage form of the present invention comprises granulating a solution of neutralized divalproex sodium and a pharmaceutically acceptable carrier; optionally further granulating the previously formed granulation with additional neutralized divalproex sodium solution to obtain an increased drug load; overcoating the granules with a hydrophobic material; blending the coated granules with a sustained release material; and formulating the resulting blend into a sustained release oral solid dosage form (e.g., tablet or capsule).

In certain preferred embodiments of the present invention, the pharmaceutically acceptable carrier comprises a plurality of particles of a material such as, for example, anhydrous lactose, dextrose or microcrystalline cellulose. A granulate is formed by spray granulating the neutralized divalproex sodium solution onto the carrier. Preferably the resulting granules are then further spray granulated with an additional solution of neutralized divalproex sodium to obtain an increased drug load. The granules are then preferably overcoated with a hydrophobic material. The sustained release material is then mixed with the coated granules. Sufficient quantities of pharmaceutically necessary tableting excipients may also be admixed with the granulate, and the resulting mixture may be compressed into tablets.

In certain embodiments, the invention is further directed to a method of treating human patients in need neutralized divalproex sustained release therapy, comprising orally administering to a human patient an effective amount of a sustained release neutralized divalproex sodium oral solid dosage form prepared in accordance with the invention on a once-a-day basis.

The invention is further related to a method of treating complex partial seizures, mania associated with bipolar disorders, and/or migraine headaches in humans comprising orally administering an effective amount of a sustained release neutralized divalproex sodium oral solid dosage form prepared in accordance with the invention to a human patient on a once-a-day basis.

In certain embodiments, the sustained release oral solid dosage forms of the present invention provide a Tₘₐₓ at from about 6 to about 20 hours after oral administration, preferably at from about 8 to about 18 hours after administration.

The term “neutralized divalproex sodium,” as used in the present invention, refers to divalproex sodium in which the valproic acid moiety has been neutralized by addition of a strong base, e.g., sodium hydroxide. Neutralized divalproex sodium is not an oligomer. Neutralized divalproex sodium also does not exhibit a 1:1 molar ratio of sodium valproate and valproic acid. The sustained release oral solid dosage forms containing divalproex sodium prepared using neutralized divalproex sodium solution, therefore, does not contain oligomeric divalproex sodium, nor does it exhibit 1:1 molar ratio of sodium valproate and valproic acid.

The term “sustained release”, as used in the present invention means that, once the drug is released from the formulation, it is released at a controlled rate such that therapeutically beneficial blood levels (but below toxic levels) of the medicament are maintained over an extended period of time from the start of drug release, e.g., providing a release over a time period, e.g., from about 8 to about 24 hours, upon exposure to an environmental fluid.

The term “environmental fluid” is meant for purposes of the present invention to encompass, e.g., an aqueous solution (e.g., an in-vitro dissolution bath) or gastrointestinal fluid.

The term “Tₘₐₓ” is meant for purposes of the present invention to mean the elapsed time from administration of a dosage form to the time the Cₘₐₓ of the medicament is achieved.

The term “Cₘₐₓ” is meant for purposes of the present invention to mean the highest plasma concentration of the drug attained within the dosing interval after single administration, e.g., about 24 hours.

The term “mean” for purposes of the present invention, when used to define a pharmacokinetic value (e.g., Tₘₐₓ) represents the arithmetic mean value measured across a patient population.

### DETAILED DISCLOSURE OF THE INVENTION

The present invention provides for a sustained release oral dosage form comprising a neutralized divalproex sodium, processes for the preparation of the sustained release oral dosage form and methods of treatment with the sustained release oral dosage form.

The sustained release oral dosage form of the present invention comprises neutralized divalproex sodium, a sustained release excipient, and other additional excipients as described herein such that the dosage form is suitable for once-a-day administration, preferably providing a release of the neutralized divalproex sodium over an about 8 to about 24 hour period upon exposure to an environmental fluid.

The sustained release oral dosage form of the present invention can be prepared in a number of ways. For example, the active agent can be incorporated in a sustained release matrix to provide for the sustained release of the active agent, a sustained release material can be coated onto an immediate release formulation comprising the active agent to provide for the sustained release of the drug, or a combination of the sustained release matrix and sustained release coating can be used. In certain preferred embodiments, the sustained release oral dosage forms of the present invention comprise granules, spheroids, or multiparticulates comprising the active agent, and which are dispersed in matrix comprising a sustained release excipient with other optional excipients such as binders, glidants, etc.

Neutralized divalproex sodium, the active agent used in the present invention, is preferably in the form of a neutralized divalproex sodium solution prepared by combining divalproex sodium with an aqueous solvent and a base. The base is added in sufficient quantities to ensure neutralization of the valproic acid moiety of the divalproex sodium. In certain preferred embodiments, the pH of the neutralized divalproex sodium solution is about 10.8±1.0, most preferably 10.8±0.5.

The base used in the present invention in the dissolution and neutralization of the divalproex sodium can be any pharmaceutically acceptable base such as sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide, mixtures thereof, and the like. A most preferred base is sodium hydroxide.

Preferably, the basic solution comprises sodium hydroxide as a base and water as an aqueous solvent. In certain embodiments, additional sodium hydroxide may be added to ensure that the valproic acid moiety of divalproex sodium is neutralized. In certain embodiments, additional water is added to the neutralized divalproex sodium solution so that the resulting solution has 20-60%, most preferably 50±3%, valproic acid activity.

In accordance with certain preferred embodiments of the present invention, the neutralized divalproex sodium solution is granulated with a pharmaceutically acceptable carrier. Granulation techniques are well known in the art and include for example, wet granulation, spray granulation and the like. Preferably, the solution of the neutralized divalproex sodium solution is spray granulated with the carrier and dried to produce divalproex sodium granules. The granules may then be sized through an appropriate sized screen, e.g., a 16 mesh screen. Alternatively, a spray coating system can be used to produce divalproex sodium pellets.

In certain preferred embodiments, the neutralized divalproex sodium solution is preferably diluted, e.g., with isopropyl alcohol before it is sprayed onto the carrier. Optionally in certain preferred embodiments, an additional amount of neutralized divalproex sodium solution is applied (e.g, sprayed) onto the granules to obtain an increased drug load.

Examples of pharmaceutically acceptable carriers for use in the present invention include, but are not limited to, calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose and mixtures thereof. Further examples of the carrier include hydroxypropylmethylcellulose, hydroxypropylcellulose, microcrystalline cellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, and suitable mixtures of the foregoing. A preferred pharmaceutically acceptable carrier is anhydrous lactose, or dextrose.

After the granules are formed, the granules are preferably overcoated with a hydrophobic material. The hydrophobic material may serve to protect the highly hygroscopic active agent from coming into contact with moisture; may serve to control the release of the active agent from the dosage form, in addition to the sustained release material; and/or may serve to increase the compressibility of the granulation comprising the active agent. Examples of hydrophobic materials which may be used in such coatings include for example, alkylcelluloses (e.g., ethylcellulose), copolymers of acrylic and methacrylic acid esters, waxes, shellac, zein, hydrogenated vegetable oil, mixtures thereof, and the like. The coating may be applied to the core using methods and techniques known to those skilled in the art. Examples of suitable coating devices include fluid bed coaters, rotor granulators, etc.

The granules (preferably coated) are then mixed with a sustained release excipient comprising a sustained release material, and formulated into a sustained release oral dosage form, e.g., by compressing the mixture into a tablet. Preferably the granules (preferably coated) are dispersed in matrix comprising the sustained release excipient.

A non-limiting list of suitable sustained-release materials which may be included in a sustained-release excipient according to the present invention include hydrophilic and/or hydrophobic materials, such as sustained release polymers, gums, acrylic resins, protein derived materials, waxes, shellac, and oils such as hydrogenated castor oil, hydrogenated vegetable oil, mixtures thereof and the like. Preferred sustained-release polymers include alkylcelluloses such as ethylcellulose, acrylic and methacrylic acid polymers and copolymers; and cellulose ethers, especially hydroxyalkylcelluloses, hydroxyalkylalkylcelluloses (especially hydroxypropylmethylcellulose) and carboxyalkylcelluloses. Preferred waxes include for example natural and synthetic waxes, fatty acids, fatty alcohols, and mixtures of the same (e.g., beeswax, carnauba wax, stearic acid and stearyl alcohol). However, any pharmaceutically acceptable hydrophobic or hydrophilic sustained-release material which is capable of imparting sustained-release of the active agent may be used in accordance with the present invention.

Prior to compressing a matrix with the active agent into a sustained release oral dosage form (e.g., a tablet), suitable quantities of additional excipients, e.g., lubricants, binders, plasticizers, granulating aids, diluents, colorants, flavorants and glidants which are conventional in the pharmaceutical art may also be included in the formulation.

Examples of lubricants for use in the present invention include magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, mixtures thereof and the like.

Examples of binders for use in the present invention include acacia, cellulose derivatives, gelatin, glucose, polyvinylpyrrolidone, sodium alginate and alginate derivatives, sorbitol, starch, mixtures thereof and the like. Binders also include hydrophillic cellulose materials, such as microcrystalline cellulose, methylcellulose, carboxymethylcellulose, mixtures thereof and the like.

Examples of suitable plasticizers for use in the present invention include water insoluble plasticizers such as dibutyl sebacate, diethyl phthalate, triethyl citrate, tributyl citrate, acetyltributyl citrate, and triacetin, acetylated monoglycerides, phthalate esters, castor oil, mixtures thereof, and the like. Acetyltributyl citrate or triethyl citrate are especially preferred plasticizers for the present invention.

Examples of glidants for use in the present invention include corn starch, silica derivatives, talc, mixtures thereof and the like.

Examples of inert diluents include calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol, sucrose, mixtures thereof and the like.

In certain preferred embodiments, the formulation further includes a pH modulating agent. The pH modulating agent is preferably an organic acid such as citric acid, tartaric acid, ascorbic acid, succinic acid, fumaric acid, malic acid, maleic acid, glutaric acid or lactic acid; acid anhydrides such as succinic anhydride and citric anhydride, and acid salts such as sodium dihydrogen phosphate, disodium dihydrogen pyrophosphate, sodium acid suphite, monopotassium citrate, potassium acid tartrate and sidium fumarate; mixtures thereof and the like.

The matrix formulation comprising the sustained release excipient, the active agent, and optional additional excipients are preferably compressed into core tablets. Thereafter the core tablets are optionally coated with a seal coating and/or a enteric coating. Additionally, a color coating may also be applied in addition to the aforementioned coatings.

In a preferred embodiment, the seal coating is applied between the tablet core and the enteric coating. Preferably the seal coating comprises a cellulose polymer, such as for example and without limitation to hydroxypropyl cellulose, hydroxypropylmethylcellulose, methoxypropyl cellulose, hydroxypropylisopropylcellulose, hydroxypropylpentylcellulose, hydroxypropylhexylcellulose, mixtures thereof, and the like. In certain embodiments, the seal coating further comprises a hydrophobic agent such as those described above (e.g., magnesium stearate). These agents preferably act as a moisture barrier to the tablet core.

The seal coating may be applied by press coating, molding, spraying, dipping and/or air-suspension or air tumbling procedures. A preferred method of applying the seal coating is by pan coating, where the seal coating is applied by spraying it onto the tablet cores accompanied by tumbling in a rotating pan. The seal coating material may be applied to the tablets as a suspension by employing solvents, e.g., an organic, aqueous, or a mixture of an organic and aqueous solvent. Examplary solvents suitable in applying the seal coating include aqueous-based solutions, an alcohol, ketone, ester, ether, aliphatic hydrocarbon, halogenated solvents, cycloaliphatic solvents, aromatic, heterocyclic, aqueous solvents, mixtures thereof, and the like. In a preferred embodiment, the seal coating comprises hydroxypropyl cellulose, hydroxypropylmethylcellulose, and magnesium stearate, and is delivered as a suspension using ethanol as a solvent.

The core tablets described above may be coated with an enteric coating to obtain delayed-release divalproex sodium tablets that remain intact in the stomach and release the active ingredient in the intestine. Suitable enteric coatings may comprise acrylic resins such as Eudragit L@, shellac, cellulose acetate butyrate, hydroxypropyl methylcellulose phthalate or combinations thereof, and the like. Additional materials suitable for use in the enteric coating include phthalates including cellulose acetyl phthalate, cellulose triacetyl phthalate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, and polyvinyl acetate phthalate, mixtures thereof, and the like.

The enteric coating like the seal coating may be applied by press coating, molding, spraying, dipping and/or air-suspension or air tumbling procedures. A preferred method of applying the enteric coating is by pan coating, where the enteric coating is applied by spraying the enteric composition onto the tablet cores accompanied by tumbling in a rotating pan. The enteric coating material may be applied to the tablet cores by employing solvents, including an organic, aqueous or a mixture of an organic and aqueous solvent. Examplary solvents suitable in applying the enteric coating include an alcohol, ketone, ester, ether, aliphatic hydrocarbon, halogenated solvents, cycloaliphatic solvents, aromatic, heterocyclic, aqueous solvents, and mixtures thereof. In a preferred embodiment, the enteric coating comprises hydroxypropylmethylcellulose phthalate.

The divalproex sodium tablet cores may further overcoated with a pharmaceutically acceptable film coating, e.g., for aesthetic purposes (e.g., including a colorant), for stability purposes (e.g., coated with a moisture barrier), for taste-masking purposes, etc. For example, the tablets may be overcoated with a film coating, preferably containing a pigment and a barrier agent, such as hydroxypropylmethycellulose and/or a polymethylmethacrylate. An example of a suitable material which may be used for such overcoating is hydroxypropylmethylcellulose (e.g., Opadry®, commercially available from Colorcon, West Point, Pa.). In a preferred embodiment, an overcoating is applied to the divalproex sodium tablets that have already been coated with a seal coating and an enteric coating. The overcoat may be applied using a coating pan or a fluidized bed, and may be applied by using a solvent, preferably an aqueous solvent. In certain preferred embodiments, the film coating and enteric coating may be combined and applied together in one step. Other excipients described herein may also be included in the enteric coating, film coating, or combination enteric/film coating such as for example, a plasticizer, glidant, lubricant, etc.

The final product is optionally subjected to a polishing step to improve the appearance of the final product and also to facilitate the manipulation of the formulation post manufacture. For example, the slippery nature of the polished dosage form aids in filling printer carrier bars with the formulation and facilitates final packaging of the product. Suitable polishing agents are polyethylene glycols of differing molecular weight or mixtures thereof, talc, surfactants (e.g., Brij types, Myrj types, glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax).

In certain alternate embodiments of the present invention, the pharmaceutically acceptable carrier may comprise a plurality of inert beads, for example, sugar beads or nonpareil seeds. The neutralized divalproex sodium solution is sprayed onto the inert beads to produce neutralized divalproex sodium coated beads, which are optionally overcoated with a hydrophobic material; thereafter the beads comprising the neutralized divalproex sodium and optionally overcoated may be further overcoated with a sustained release excipient to provide for the sustained release of the divalproex sodium, and then formulated into oral solid dosage forms, such as capsules or tablets. In certain embodiments, the sustained release divalproex sodium coated beads may additionally be coated with an enteric coating after the sustained release excipient is applied. In yet another embodiment, a seal coating may be applied to the drug containing beads, after the application of the sustained release excipient prior to the application of the enteric coating. After the coatings are applied, the beads may be admixed with sufficient quantities of pharmaceutically necessary tableting excipients and formulated into oral solid dosage forms, e.g., capsules or tablets).

In a preferred embodiment of the invention, the neutralized divalproex sodium solution is sprayed onto the pharmaceutically acceptable carrier in a fluid bed processor. The divalproex sodium granules may then be dried and then sifted using a mesh screen, e.g., with a 16 mesh screen, to produce divalproex sodium granules. Alternatively, a spray coating system can be used to produce divalproex sodium pellets.

The sustained release oral solid dosage forms of the present invention (e.g., tablets) produced as describe herein do not contain divalproex sodium that is an oligomeric compound and does not have a 1:1 molar ratio of sodium valproate and valproic acid. Rather, the sustained release oral solid dosage forms of the present invention contain divalproex sodium in which the valproic acid moiety has been neutralized.

### DETAILED DESCRIPTION OF THE PREFERRED EMBODIMENTS

The following examples illustrate various aspects of the present invention. They are not to be construed to limit the claims in any manner whatsoever.

### EXAMPLE 1

In Example 1, sustained release pH modified divalproex sodium tablets were prepared having the following formulation in Table 1 below:

TABLE 1

| Ingredient | Function | mg/tablet | % w/w |
|---|---|---|---|
| Core Tablets | | | |
| Sodium Valproate^{(a)} | Active | 576.24 | 51.73 |
| Sodium Hydroxide, NF | pH-modifier | ★ | ★ |
| Sugar Compressible (Di-PAC ®) | Filler | 60.96 | 5.47 |
| Ethylcellulose, NF (Ethocel | Polymer/Film-former | 72.41 | 6.50 |
| 100 cps) Standard Premium | | | |
| Acetyltributyl Citrate, USP | Plasticizer | 14.48 | 1.30 |
| (ATBC) | | | |
| Purified Water, USP | Solvent | ★★ | ★★ |
| Acetone, NF | Solvent | ★★ | ★★ |
| Isopropyl Alcohol, USP | Solvent | ★★ | ★★ |
| Hydroxypropyl Methylcellulose, | Dissolution-rate | 278.50 | 25.00 |
| USP (Methocel K100M Premium CR) | controlling hydrogel | | |
| | matrix | | |
| Glyceryl Monostearate, NF | Anti-adherant | 100.26 | 9.00 |
| Magnesium Stearate, NF/FCC | Lubricant | 11.14 | 1.00 |
| Sub-total | | 1114.00 | 100.00^{(b)} |
| Seal Coating | | | |
| Hydroxypropyl Methylcellulose, | Film Former | 13.78 | 1.20 |
| USP (HPMC E5) | | | |
| Hydroxypropyl Cellulose, NF | Film Former | 13.78 | 1.20 |
| (Klucel EF) | | | |
| Magnesium Stearate, NF/FCC | Hydrophobic agent | 6.89 | 0.60 |
| Ethanol- SDA 3A 190 Proof | Solvent | ★★ | ★★ |
| Sub-total: | | 1148.45 | 3.00^{(b)} |
| Enteric and Color Coating | | | |
| Hypromellose 55, NF, Substitution | Enteric polymer | 12.54 | 1.07 |
| Type 200731 | | | |
| Opadry Gray (YS-1-17506-A) | Colorant | 5.98 | 0.51 |
| Triethyl Citrate, PG/NF (TEC) | Plasticizer | 1.17 | 0.10 |
| Talc, USP | Anti-sticking agent | 3.75 | 0.32 |
| Isopropyl Alcohol, USP | Solvent | ★★ | ★★ |
| Purified Water, USP | Solvent | ★★ | ★★ |
| Sub-total | | 1171.89 | 2.00^{(b)} |
| Total | | 1171.89 | |

★Less than 0.01% (w/w) is used to adjust the pH of solution to 10.8
★★Evaporated during processing
^{(a)}Core tablets contain neutralized divalproex sodium, equivalent to 576.24 mg of sodium valproate, or 500 mg of valproic acid activity.
^{(b)}Total percentage for the specific step.

Process:

Divalproex sodium (pH-modified) solution was prepared by dissolving sodium valproate in water followed by adjustment of the pH of solution to be more than 10.8 with sodium hydroxide. Divalproex sodium (pH-modified) active I pellets were made through wet granulation process by spraying the divalproex sodium (pH modified solution that had been diluted with isopropyl alcohol onto Di-Pac®. In order to obtain higher drug loading, active II pellets were made by spraying the divalproex sodium (pH-modified) solution over active I pellets after dilution with IPA. These processes could not be combined into one step due to the restriction in the minimum and maximum load capacity of the fluidized-bed granulator.

Divalproex sodium (pH-modified) active II pellets were subsequently coated with ethylcellulose. The core tablets were obtained by compressing a blend comprising of the EC-coated active pellets with hydroxypropyl methylcellulose (HPMC K100M CR) and other excipients.

A seal-coating layer of hydroxypropylcellulose (Klucel EF) and magnesium stearate was applied to further protect the core tablets from gaining moisture. Finished products were obtained by applying the enteric polymer (HPMCP 55) and the colorant (Opadry Gray) onto seal-coated tablets in a one-step coating process in an O'Hara perforated coating pan.

### EXAMPLE 2

In Example 2, sustained release pH modified divalproex sodium tablets were prepared having the following formulation in Table 2 below:

TABLE 2

| Ingredient | Function | mg/tablet | % w/w |
|---|---|---|---|
| Core Tablets | | | |
| Sodium Valproate^{(a)} | Active | 576.24 | 44.60 |
| Sodium Hydroxide, NF | pH-modifier | ★ | ★ |
| Sugar Compressible (Di-PAC ®) | Filler | 60.33 | 4.67 |
| Ethylcellulose, NF (Ethocel | Polymer/Film-former | 78.55 | 6.08 |
| 100 cps) Standard Premium | | | |
| Acetyltributyl Citrate, USP | Plasticizer | 15.76 | 1.22 |
| (ATBC) | | | |
| Purified Water, USP | Solvent | ★★ | ★★ |
| Acetone, NF | Solvent | ★★ | ★★ |
| Isopropyl Alcohol, USP | Solvent | ★★ | ★★ |
| Hydroxypropyl Methylcellulose, | Dissolution-rate | 369.51 | 28.60 |
| USP (Methocel K100 M Premium CR) | controlling hydrogel | | |
| | matrix | | |
| Citric Acid Anhydrous, USP Fine | pH-modulator | 115.38 | 8.93 |
| Granular | | | |
| Povidone, USP (Plasdone K-30) | Binder | 32.30 | 2.50 |
| Microcrystalline Cellulose | Binder | 32.30 | 2.50 |
| (Avicel PH-102) | | | |
| Magnesium Stearate, NF/FCC | Lubricant | 11.63 | 0.90 |
| Sub-total | | 1292.00 | 100.00^{(b)} |
| Seal Coating | | | |
| Hydroxypropyl Methylcellulose, | Film Former | 15.98 | 1.20 |
| USP (HPMC E5) | | | |
| Hydroxypropyl Cellulose, NF | Film Former | 15.98 | 1.20 |
| (Klucel EF) | | | |
| Magnesium Stearate, NF/FCC | Hydrophobic agent | 7.99 | 0.60 |
| Ethanol- SDA 3A 190 Proof | Solvent | ★★ | ★★ |
| Sub-total: | | 1331.95 | 3.00^{(b)} |
| Enteric and Color Coating | | | |
| Hypromellose 55, NF, | Enteric polymer | 44.68 | 3.22 |
| Substitution Type 200731 | | | |
| Opadry Gray (YS-1-17506-A) | Colorant | 20.81 | 1.50 |
| Triethyl Citrate, PG/NF (TEC) | Plasticizer | 4.44 | 0.32 |
| Talc, USP | Anti-sticking agent | 13.32 | 0.96 |
| Isopropyl Alcohol, USP | Solvent | ★★ | ★★ |
| Purified Water, USP | Solvent | ★★ | ★★ |
| Sub-total | | 1415.20 | 4.00^{(b)} |
| Total | | 1415.20 | |

★Less than 0.01% (w/w) is used to adjust the pH of solution to 10.8
★★Evaporated during processing
^{(a)}Core tablets contain neutralized divalproex sodium, equivalent to 576.24 mg of sodium valproate, or 500 mg of valproic acid activity.
^{(b)}Total percentage for the specific step.

Process:

The sustained release oral dosage form Example 2 was prepared in accordance with the process of Example 1.

### EXAMPLE 3

Sustained release formulations prepared in accordance with Examples 1 and 2 were compared to a reference standard Depakote® ER in a three-way crossover study under both fed and fasting conditions. The results of the study are summarized in Table 3 below for the formulations prepared in accordance with Example 1 and in Table 4 below for the formulations prepared in accordance with Example 2.

TABLE 3

| | | Test Values | | Reference Values | | | | 90% |
|---|---|---|---|---|---|---|---|---|
| | | (Ex. 1 formulation) | | (Depakote ER) | | G-Mean | | Confidence |
| Condition | Parameters | Mean | CV (%) | Mean | CV (%) | Ratio* | CV (%) | Intervals |
| Fasting | Cₘₐₓ | 27.58 | 28.91 | 25.23 | 38.04 | 1.124 | 15.49 | 97.5-126 |
| | AUC₀₋ₜ | 829.35 | 39.02 | 698.59 | 48.82 | 1.238 | 32.23 | 107-142 |
| | Tₘₐₓ | 12.75 | 26.10 | 14.75 | 46.23 | 0.942 | 66.36 | |
| Fed | Cₘₐₓ | 23.28 | 24.77 | 25.01 | 15.04 | 0.942 | 21.15 | 77.3-108 |
| | AUC₀₋ₜ | 682.38 | 29.73 | 674.02 | 23.90 | 1.027 | 11.69 | 90.9-110 |
| | Tₘₐₓ | 15.44 | 43.21 | 20.89 | 32.86 | 0.712 | 45.35 | |

*Log transformed analysis

TABLE 4

| | | Test Values | | Reference Values | | | | 90% |
|---|---|---|---|---|---|---|---|---|
| | | (Ex. 2 formulation) | | (Depakote ER) | | G-Mean | | Confidence |
| Condition | Parameters | Mean | CV (%) | Mean | CV (%) | Ratio* | CV (%) | Intervals |
| Fasting | Cₘₐₓ (μ/mL) | 29.20 | 19.33 | 25.23 | 38.04 | 1.209 | 31.42 | 104-135 |
| | AUC₀₋ₜ | 874.51 | 30.05 | 698.59 | 48.82 | 1.335 | 29.82 | 114-152 |
| | (μ · hr/mL) | | | | | | | |
| | Tₘₐₓ(hr) | 11.38 | 15.54 | 14.75 | 46.23 | 0.861 | 73.21 | |
| Fed | Cₘₐₓ (μ/mL) | 27.41 | 30.17 | 25.01 | 15.041 | 1.058 | 27.17 | 89.6-125 |
| | AUC₀₋ₜ | 737.79 | 26.05 | 674.02 | 23.90 | 1.086 | 14.70 | 98.9-119 |
| | (μ · hr/mL) | | | | | | | |
| | Tₘₐₓ (hr) | 14.33 | 52.79 | 20.89 | 32.86 | 0.623 | 51.84 | |

*Log transformed analysis

Many other variations of the present invention will be apparent to those skilled in the art and are meant to be within the scope of the claims appended hereto.

1. A process for preparing a sustained release oral dosage form of neutralized divalproex sodium comprising
(a) preparing a neutralized divalproex sodium solution by combining divalproex sodium, having a sodium valproate moiety and a valproic acid moiety, with a base and an aqueous solvent, the base being added in sufficient amount to ensure neutralization of the valproic acid moiety of the divalproex sodium;
(b) combing the neutralized divalproex sodium solution with a pharmaceutically acceptable carrier to form a neutralized divalproex sodium composition; and
(c) mixing the composition with a sustained release material to provide for the sustained release of the neutralized divalproex over an 8 to 24 hour period upon exposure to an environmental fluid.

2. The process of claim 1, wherein step (b) comprises granulating the neutralized divalproex sodium solution with the pharmaceutically acceptable carrier to form a neutralized divalproex sodium granulation.

3. The process of claim 2, further comprising overcoating the granules with a coating comprising a hydrophobic material prior to mixing the granules with the sustained release excipient.

4. The process of claim 2, wherein said granules are dispersed in a matrix comprising said sustained release excipient.

5. The process of claim 4, wherein said matrix further comprise an additional excipient.

6. The process of claim 5, wherein the excipient is selected from the group consisting of a lubricant, a disintegrant, a binder, a glidant, an inert diluent, a pH modulating agent and mixtures thereof.

7. The process of claim 2, wherein prior to mixing with the sustained release excipient, the granules are dried to evaporate any excess solvent, and thereafter screened to obtain uniformly sized particles.

8. The process of claim 4, further comprising compressing the matrix into a core tablet.

9. The process of claim 8, further comprising applying a seal coat to the core tablets.

10. The process of claim 8, further comprising applying an enteric coat to the core tablet.

11. The process of claim 1, wherein said base is selected from the group consisting of sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide and mixtures thereof.

12. The process of claim 1, wherein said base is sodium hydroxide.

13. The process of claim 1, wherein said granulation is spray granulation.

14. The process of claim 1, wherein said sustained release material is selected from the group consisting of a sustained release polymer, a gum, an acrylic resin, a protein derived material, a wax, shellac, an oil, and mixtures thereof.

15. The process of claim 1, wherein said sustained release material is hydroxypropylmethylcellulose.

16. The process of claim 3, wherein said hydrophobic material is ethylcellulose.

17. The process of claim 1, wherein the pharmaceutically acceptable carrier comprises a plurality of substrates.

18. The process of claim 17, wherein said substrates are sprayed with the neutralized divalproex sodium solution to obtain divalproex sodium coated substrates.

19. The process of claim 17, wherein said substrates are inert beads.

20. The process of claim 10, wherein said enteric coat comprises an acrylic resin.

21. The sustained release oral dosage form of claim 1.

22. A sustained release oral dosage form of neutralized divalproex sodium comprising neutralized divalproex sodium and a sustained release material to provide for the sustained release of the neutralized divalproex over an 8 to 24 hour period upon exposure to an environmental fluid.

23. A method of treatment with neutralized divalproex therapy comprising:
orally administering the sustained release oral dosage form of claim 21 to a patient in need of treatment with sustained release neutralized divalproex therapy.
